(19) United States
(12) Patent Application Publication
(10) Pub. No.: US 2026/0131064 A1
(43) Pub. Date: May 14, 2026

(54) CANNULA ASSEMBLY

(71) Applicant: Rheinisch-Westfälische Technische Hochschule Aachen, abgekürzt RWTH Aachen Körperschaft des öffent, Aachen (DE)

(72) Inventors: Jan HEYER, Aachen (DE); Franziska SCHUBERT, Aachen (DE); Thorsten ORLIKOWSKY, Aachen (DE); Thomas SCHMITZ-RODE, Aachen (DE); Mark SCHOBERER, Aachen (DE); Ulrich STEINSEIFER, Aachen (DE)

(21) Appl. No.: 19/120,076

(22) PCT Filed: Sep. 14, 2023
(86) PCT No.: PCT/EP2023/075282

§ 371 Date: Apr. 10, 2025

(30) Foreign Application Priority Data
Oct. 20, 2022 (DE) 10 2022 127 775.5

(51) Int. Cl. A61M 1/36; A61F 2/90; A61M 5/158

(52) U.S. Cl. CPC A61M 1/3659; A61F 2/90; A61M 5/158; A61M 2005/1585; A61M 2005/1586; A61M 2240/00

(57) ABSTRACT

A cannula assembly is provided for insertion into the umbilical cord of a newborn, at the distal end region of which cannula assembly a distally tapering insertion region is disposed, next to which, in the direction toward proximal, a cannula is disposed, at the distal end region of which a crimped, expandable stent is disposed, the outside diameter of which when crimped is preferably smaller than or equal to the outside diameter of the cannula, and the inside diameter of which when expanded is preferably greater than or equal to the inside diameter of the cannula. Also provided is a set comprising such a cannula assembly and at least two inner cannulas having different tip designs.

The invention relates to a cannula assembly for insertion into the umbilical cord of a newborn.

Among newborns, and especially premature babies, there may be a need, for example, to steadily medicate or to form an extracorporeal blood circulation system to support bodily functions, for example for extracorporeal membrane oxygenation or also to support kidney function or other uses.

Generally, access to the blood circulation system via the umbilical cord is an obvious choice for newborns; this, however, proves to be problematic since the umbilical cord is closed off on its own shortly after birth due to a natural vasospasm and can then no longer be used.

Against this background, it is an object of the invention to provide a cannula assembly by way of which the umbilical cord can be cannulated, not just when it is being supplied with blood (that is to say, before being cut), but preferably can be re-opened even after being cut and after the onset of the vasospasm and be used as an access point, for example, to implement an extracorporeal circulation system, or for another use, such as medication.

According to the invention, this object is achieved by a cannula assembly, at the distal end region of which a distally tapering insertion region is disposed, next to which, in the direction toward proximal, a cannula is disposed, at the distal end region of which a crimped, expandable stent is disposed.

In common parlance, distal and proximal are understood to mean directional information with respect to a user of the cannula assembly when the cannula assembly is used as intended. Distal, from the view of the user, thus signifies in the direction toward the umbilical cord, and proximal denotes the opposite direction, which is to say, in the direction toward the user.

The cannula disposed toward proximal, next to the insertion region, is thus located behind the insertion region, when viewed from distal to proximal.

During use of the cannula assembly, the invention allows for the possibility of carrying out dilation of the umbilical cord by way of the tapered insertion region, which facilitates the insertion of the cannula, with the distally disposed, crimped stent thereof, into the umbilical cord. Following expansion of the previously crimped stent, the umbilical cord is held open by the stent, which is to say, the vasospasm is overcome so that the umbilical cord can be permanently utilized as an access point.

The invention can preferably provide that the tapering insertion region is designed so as to taper evenly over the entire circumference. The insertion region thus forms a preferably rotation-symmetrical region. However, the tapering may also take place unevenly so that no rotational symmetry is present in the insertion region.

In one possible embodiment, the stent at the distal end region of the cannula can adjoin the end of the cannula, and in particular can be attached thereto. In this embodiment, the end of the cannula does not project into the stent. However, it may also be provided that the end of the cannula is located in the stent. The stent can thus be attached to the outside of the cannula surface. The end region of the cannula can be located on the inside in an axial sub-region of the stent or can completely extend through the stent to the distal end thereof. Preferably, however, it is provided that the cannula does not protrude toward distal beyond the end of the stent.

If the end region of the cannula is at least partially located in the stent, this end region of the cannula is compressed by the stent when the stent is crimped. The cannula is accordingly flexible or suitably compressible and is preferably itself designed so as to be expandable when the stent is (being) expanded.

According to one possible embodiment, the stent is coated with a foil over the entire or a part of the length thereof. The coating can be provided on the outside or inside or on both sides of the stent. A foil, preferably on the inner side, offers an ideally seamless connection or transition into the inner lumen of the cannula located further proximal. The foil thus preferably transitions into the inner surface of the cannula.

When an outside foil is provided, this ideally represents the seamless connection to the outer surface of the cannula.

Such an inner or outer foil can, for example, be formed by an end region of the cannula which has a reduced thickness. The foil and cannula can thus, for example, be made of the same material and preferably be designed in one piece. In another embodiment, the stent can also be embedded in the material thickness of the cannula.

According to one possible embodiment, the outside diameter of the stent, when crimped, is smaller than or equal to the outside diameter of the cannula, in particular in comparison with the region of the cannula that is not covered by the stent. More preferably, the inside diameter of the stent, when expanded, is greater than or equal to the inside diameter of the cannula.

This opens up the advantage that elements that also fit through the cannula can be moved through the stent. Such elements can be, for example, removable elements of the cannula assembly.

In preferably all embodiments of the invention, the cannula forms a structural unit together with the stent, which is to say, the stent and the cannula are joined, and in particular inseparably joined when used as intended.

In a first possible embodiment, the cannula assembly according to the invention can have a one-piece design, in particular at least with respect to the components thereof, these being the insertion region, the stent and the cannula. In this case, the tapering insertion region, which is disposed, in the direction toward distal, next to the stent disposed at the cannula, is an integral portion of the preferred unit made up of the stent and the cannula.

In a second possible embodiment, the cannula assembly can have a multi-piece design, in particular within the meaning that the tapering insertion region is formed separately from the unit that is made up of the stent and the cannula, in particular at a separate element, which can preferably be removed from, or out of, the cannula assembly, for example, after insertion into the umbilical cord, in particular through the expanded stent and the cannula.

With respect to the aforementioned first option of the embodiment, the invention can thus provide that the tapering insertion region is preferably at least regionally formed by the stent itself at the distal end region of the cannula, for which purpose the stent, when crimped, tapers at least regionally toward distal. The stent does not have to taper over the entire extension thereof, but at least at the distal region thereof. The stent can thus comprise a region that does not have a tapering cross-section and a tapering region distally adjoining the same. The stent can also be designed so as to taper throughout.

In this first embodiment, due to the taper thereof when crimped, the stent thus assumes, on the one hand, the function of supporting the widening of the umbilical cord during the insertion process of the cannula assembly and, on the other hand, thereafter assumes the further function, after the expansion thereof, of overcoming the vasospasm and holding the umbilical cord open.

In a preferred embodiment, it may be provided that the stent, when expanded, has a constant cross-section in the extension direction thereof, and in particular has an inner clear cross-section that is greater than or equal to the inner cross-section of the cannula. From such an expanded state having a constant cross-section, the stent can thus be converted into the crimped state by the intensity of the crimping increasing toward distal.

Another specific embodiment of the first embodiment of a one-piece cannula assembly can also provide that a distally tapering insertion region is fixedly disposed next to the crimped and expandable stent, in the direction toward distal. This insertion region is joined to the stent. This insertion region can, for example, be formed by a sub-region of the cannula, which extends through the stent and protrudes from the same toward distal, or by a tapering component, which is distally attached to the stent.

In the second possible specific embodiment, which is preferred for the invention, it may also be provided that the tapering insertion region is formed by the distally tapering end, having a thickened cross-section, of a rod-shaped or tubular insertion aid, onto which the cannula, together with the stent, is pushed, or at least can be pushed, so as to point in the direction toward the thickened end.

In a cannula assembly in which the unit made up of the stent and the cannula is pushed onto such an insertion aid, the stent is thus preferably located, in the direction toward proximal, next to the thickened end, and in particular adjoining the same, preferably at a distance, and thus, in particular, between the thickened end and the cannula, or at least in front of the proximal end region thereof. Viewed from distal to proximal, the stent is thus located behind the thickened end.

The thickened end of the insertion aid preferably has a tapering design toward distal and toward proximal, wherein the taper toward distal preferably takes place with a lesser inclination compared to the taper toward proximal. In a tubular insertion aid, it may be provided that the thickened end is tapered in both directions up to an identical cross-section in front of and behind the thickened end. The tubular design of the insertion aid has the advantage that further elements can be moved through the same, or that this aid can also be utilized for supplying pharmaceutical drugs during the insertion process.

An insertion aid of this specific embodiment can thus be temporarily utilized in the cannula assembly, namely for the purpose of facilitating the insertion of the unit made up of the cannula and the stent into the umbilical cord. For this purpose, this unit is pushed onto the insertion aid, for example, until the stent arrives at, and in particular comes to bear against, the proximal side of the thickened end, and the overall cannula assembly thus formed can be inserted into the umbilical cord with the thickened and distally tapering end of the insertion aid first.

According to a preferred refinement, the maximum outside diameter of the insertion aid at the thickened end is smaller than the inside diameter of the cannula and/or of the expanded stent. This creates the option of being able to remove the insertion aid from the cannula assembly, after the stent has been expanded, through the stent and through the cannula, in the direction toward proximal.

More preferably, it is provided that the outside diameter of the crimped stent is smaller than the maximum outside diameter of the insertion aid. This ensures that the unit made up of the cannula and the stent cannot be inadvertently guided over the thickened end during insertion.

All possible specific embodiments, which is to say, in particular the first and second specific embodiments of the kind described, can provide that the cannula and the stent disposed in the distal end region thereof are surrounded by a sleeve that can be pulled over the cannula toward proximal, which holds the stent in the crimped state, wherein the stent, after the sleeve has been removed, and preferably pulled back, can automatically expand, in particular into an expanded state in which the stent has a constant cross-section in the extension direction thereof. Such a sleeve can thus assume the function of a retaining element, which holds the stent in a crimped state. Such a sleeve can be provided in addition to the above-described outer sleeve or form this described sleeve.

In particular in the aforementioned type of the first embodiment, it may be provided that the cross-section of the sleeve tapers toward distal. This, however, is also possible in the other embodiments.

If such a sleeve is provided, the sleeve can furthermore preferably have at least one weakened line extending in the axial direction of the cannula/sleeve, at which the sleeve can be torn open. Such a weakened line is preferably disposed at least in a region of the sleeve having a distally tapering cross-section, if such a region is formed.

In particular when the stent is not designed so as to expand automatically, the invention may provide, in another preferred refinement, that an expandable balloon is disposed beneath the stent on the rod-shaped or tubular insertion aid, the inner volume of which can be filled with a fluid, wherein the stent can be converted into the expanded state by having a force applied to the inside by way of the balloon. For this purpose, the insertion aid, proximally next to the thickened end, comprises such a balloon, for example an inflatable sleeve, which surrounds a region proximal in front of the thickened end over 360 degrees and can be inflated from the inside out. Pushing the unit made up of the cannula and the stent moves the balloon beneath the stent, so that the inflation of the balloon converts the stent into the expanded state.

It is preferably provided that the balloon is attached to or around the insertion aid, and the inner volume thereof can be filled with a fluid through a channel in the insertion aid, in particular a channel within the wall thickness of the tubular insertion aid.

The insertion aid is preferably designed so as to be removable, together with the balloon, in the unfilled or emptied state, through the stent and through the cannula, in the direction toward proximal, from the cannula assembly.

As mentioned above, the insertion aid can preferably have a tubular design. In such a case, a connecting element is preferably disposed at the proximal end of the insertion aid, preferably a Luer lock, for supplying and/or discharging liquids from a vessel having a corresponding connecting element.

A connecting element, for example a Luer lock, can preferably also be provided at the cannula, and in particular at the proximal end thereof.

Further preferably, the cannula, and in particular a proximal end region of the cannula, which is preferably pushed in up to just before the umbilical cord, can comprise a kink protector, which is designed, for example, as a spiral reinforcement.

In one refinement, it may also be provided that the cannula assembly according to the invention comprises a rigid or flexible inner cannula as a further component, which is designed so as to be insertable into the tubular insertion aid or, conversely, in such a way that the insertion aid can be pushed on, preferably comprising the pushed-on unit made up of the cannula and stent. Such an inner cannula preferably has a constant cross-section over the entire extension, and in particular a round cross-section.

It may thus be provided during use that the inner cannula initially pierces and is inserted as a guiding aid into an umbilical cord. For example, the inner cannula can straighten an umbilical cord. The insertion aid comprising the pushed-on unit made up of the cannula and the stent can then be pushed onto the inner cannula then located in the umbilical cord and can be pushed toward the umbilical cord until the insertion aid penetrates into the umbilical cord, with the tapering, thickened end, and widens the same, so that the stent and cannula can also enter. Thereafter, the inner cannula can be removed again, and the insertion aid can also be removed after the stent has been expanded.

The inner cannula itself preferably also has a tapering tip or a tapering distal end. It may be provided that the distal end of the inner cannula is designed to be edged and, in particular, as a blade, or blunt, and preferably convex. An end designed as a blade offers the added advantage of being able to pierce the umbilical cord wall more easily with the inner cannula.

The invention can preferably also provide a set comprising a cannula assembly of the above-described type, in particular comprising a unit made up of a cannula and a stent, a tubular insertion aid, and at least two inner cannulas having different designs for the distal end. Using such a set, a user can then select from multiple options for various possible circumstances, in particular for the curves of the umbilical cord, by selecting a suitable inner cannula from the plurality in the set. The invention preferably provides that the separate elements of the invention are all disposed, or can be disposed, around a shared center line. These separate elements include a) the unit made up of the cannula and the stent, b) the insertion aid, and c) the inner cannula.

Specific embodiments of the invention will be described in more detail based on the figures.

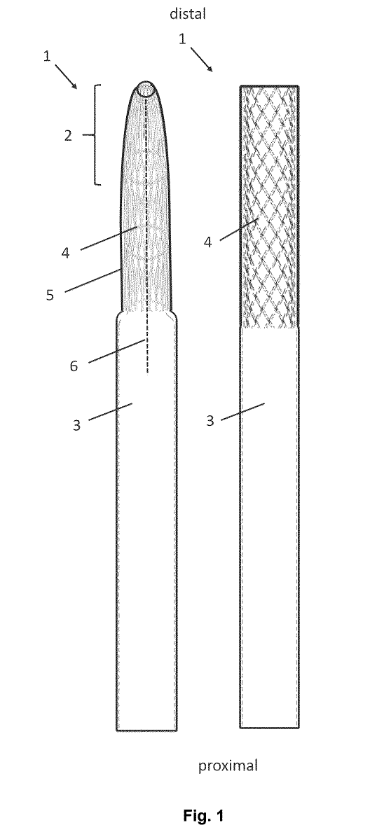

FIG. 1 shows the aforementioned first specific embodiment in one possible variant.

The cannula assembly 1 according to the invention has a distally tapering insertion region 2 at the distal end (here, at the top), and a crimped, expandable stent 4 located proximally before the same, which is connected to a cannula 3.

The distal region of the stent 4 itself, here, forms the tapering insertion region 2, for example, by the stent 4 being crimped more strongly in the distal region thereof than in the region located proximally therebefore.

The stent 4 here is furthermore coated with a foil 5, which can preferably at least regionally also be located over the cannula 3. This foil 5 can also be used to hold the stent 4 in the crimped state, for which purpose the foil is then also tapered in the cross-section toward distal.

The stent 4 can thus be self-expanding when the foil 5 is being removed. For this purpose, the foil can, for example, be pulled toward proximal. So as to facilitate this, the foil 5 can have a line-shaped weakening 6 so that the foil 5 tears open more easily.

The left illustration of FIG. 1 shows the cannula assembly 1 while the stent 4 is still crimped, which is to say, prior to the insertion into an umbilical cord. The right illustration shows the same cannula assembly after the foil 5 has been removed and the expansion of the stent 4 has been generated thereby. The cannula assembly assumes this state when it is inserted into an umbilical cord. The insertion into the umbilical cord is facilitated by the tapering crimped stent 4, wherein the umbilical cord is held open by the expanded stent 4, and the vasospasm is overcome.

In this first embodiment, the cannula assembly 1 is a one-piece assembly as it relates to the components that are the insertion region 2, the cannula 3, and the stent 4.

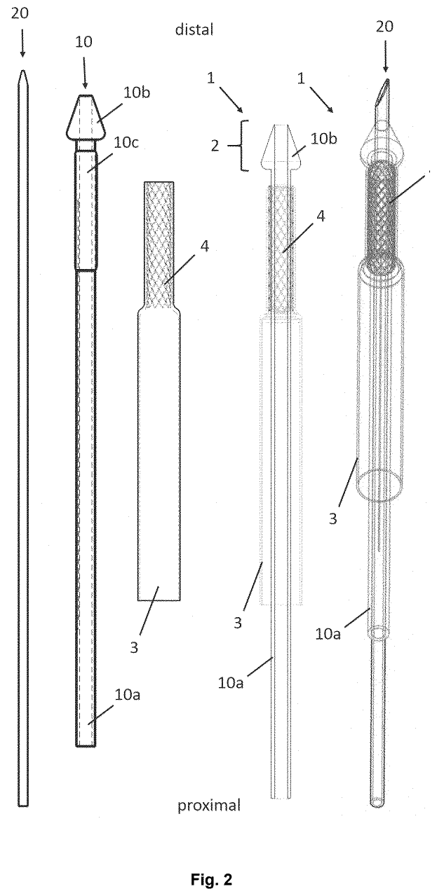

FIG. 2 shows the aforementioned second embodiment, in which the insertion region 2 is formed at a separate element, namely at an insertion aid 10.

Similarly to FIG. 1, the cannula 3 and the stent 4 are also designed as a joined unit here. The stent 4 can be coated with a foil on the inside, outside or on both sides; however, this is not mandatory. FIG. 2 shows the stent 4 in the crimped state in all partial illustrations.

The insertion aid 10 essentially represents a tube 10a having a preferably constant inner and outer cross-section throughout. Distally, the tube 10a or the insertion aid 10 comprises a thickened end region 10b, which tapers toward distal and forms the insertion region 2, by way of which the insertion into the umbilical cord is facilitated. An inflatable balloon 10c is provided proximally in front of or next to the thickened end region 10b. The balloon 10c can be filled by way of a line in the wall thickness of the tube 10a.

The design is selected such that the unit made up of the cannula 3 and the stent 4 can be pushed onto the insertion aid so that the balloon 10c is positioned beneath the crimped stent. In this way, the cannula assembly 1 is formed in one possible variant of the second specific embodiment.

A further variant can provide that the cannula assembly 1 comprises another separate component, namely an inner cannula 20 made of a flexible or rigid element, and in particular a rod-shaped element having a tapering distal end, which can be designed, for example, as a blade. The inner cannula has a hollow or solid design.

This inner cannula 20 is designed so as to be able to be inserted into the assembled cannula assembly made up of the unit comprising the cannula/stent and the insertion aid, and in particular into the interior of the tube 10a of the insertion aid 10.

In this way, the cannula assembly 1 according to the invention, on the far right in FIG. 2, can be formed. During use, it may be provided that, initially, the inner cannula 20 pierces and is inserted into the umbilical cord, and that, thereupon, the insertion aid 10, comprising the unit made up of the cannula and stent placed thereon, is pushed so as to glide on the inner cannula.

When the suitable position has been found, the inner cannula 20 may be removed or may initially remain in place. The balloon 10c is inflated, whereby the stent 4 is expanded and, thereafter, the insertion aid 10 can be pulled out of the unit made up of the cannula 3 and the stent 4 toward proximal (and possibly the remaining inner cannula 20 as well).

A blood connection to the blood circulation system of a newborn can then be established via the hollow interior of the cannula 3 and the stent 4, which holds the umbilical cord open.

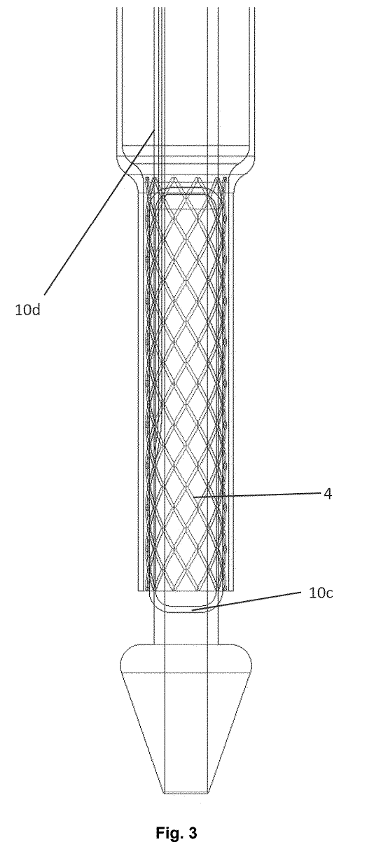

FIG. 3 shows a detailed illustration of the distal end region of a cannula assembly according to the invention having the aforementioned design details.

1. A cannula assembly configured for insertion into the umbilical cord of a newborn, comprising: a distally tapering insertion region disposed a distal end region of the cannula assembly; a cannula disposed next to the distally tapering insertion region in a direction toward a proximal end of the cannula assembly; and a crimped, expandable stent disposed at a distal end region of the cannula, an outside diameter of the crimped, expandable stent, when not expanded being smaller than or equal to an outside diameter of the cannula, and an inside diameter of the crimped, expandable stent, when expanded, being greater than or equal to an inside diameter of the cannula

2. The cannula assembly according to claim 1, wherein at least a region of the distally tapering insertion region is formed by the crimped, expandable stent at the distal end region of the cannula, at least regions of the crimped, expandable stent, when not expanded, tapering toward a distal end thereof, the crimped, expandable stent, when expanded, having a constant cross-section in an extension direction thereof.

3. The cannula assembly according to claim 1, wherein the distally tapering insertion region is formed by distally tapering end region having a thickened cross-section end region, of a rod-shaped or tubular insertion aid onto which the cannula together with the crimped, expandable stent is pushed, or configured to be pushed, so as to point in a direction toward the distally tapering end region having the thickened cross-section end region.

4. The cannula assembly according to claim 3, wherein a maximum outside diameter of the insertion aid at the thickened cross-section end region is smaller than the inside diameter of the cannula and/or of the crimped, expandable stent when expanded so that the insertion aid, after the crimped, expandable stent has been expanded, can be removed through the expanded crimped, expandable stent and through the cannula in the-a direction toward proximal of the cannula assembly, out of the cannula assembly, the outside diameter of the crimped, expandable stent when not expanded being smaller than a maximum outside diameter of the insertion aid

5. A The cannula assembly according to claim 2, wherein the cannula and the crimped, expandable stent are surrounded by a sleeve which is configured to be pulled over the cannula toward the proximal end of the cannula assembly and to hold the crimped, expandable stent when not expanded, the crimped, expandable stent, after the sleeve has been removed, and pulled back, being automatically expandable, and, when expanded, having the constant cross-section in the extension direction thereof.

6. A cannula assembly according to claim 2 or 5, wherein a cross-section of the sleeve tapers toward a distal end of the cannula assembly.

7. The cannula assembly according to claim 5, wherein the sleeve has at least one weakened line extending in an axial direction of the cannula and the sleeve at which the sleeve can be torn open, the at least one weakened line being disposed at least in a region of the sleeve which has a distally tapering cross-section.

8. A cannula assembly according to claim 3, wherein an expandable balloon is disposed beneath the stent on the rod-shaped or tubular insertion aid, an inner volume of the expandable balloon being fillable with a fluid, the crimped, expandable stent being expanded by having a force applied to an inside thereof by way of the balloon.

9. The cannula assembly according to claim 8, wherein the balloon is attached to the insertion aid and the inner volume thereof can be filled with a fluid through a channel within the wall thickness of the tubular insertion aid the insertion aid being removable from the cannula assembly, together with the balloon, in the unfilled or re-emptied state, through the crimped, expandable stent and through the cannula, in the direction toward the proximal end of the cannula assembly.

10. A cannula assembly according to claim 3, the insertion aid is tubular.

11. The cannula assembly according to claim 10, Luer lock is disposed at a proximal end of the insertion aid, for supplying and/or discharging liquids from a vessel having a corresponding connecting element.

12. The cannula assembly according to claim 10 or 11, further comprising a rigid or flexible inner cannula configured to be inserted into the tubular insertion aid and/or so configured that there onto the cannula assembly can be pushed with the tubular insertion aid after the inner cannula has been inserted into an umbilical cord, the inner cannula having a tapering tip.

13. The cannula assembly according to claim 12, wherein a distal end of the inner cannula is edged, as a blade, or is blunt, and, furthermore, is convex.

14. A set comprising a cannula assembly according to claim 12, comprising at least two of the inner cannula, each of the inner cannulas being configured differently at the distal end thereof.
